SEQUENCE LISTING (10 sequences)

SEQ ID NO: 1 - DNA, 20 bp, Escherichia virus T7
  source 1..20: mol_type = genomic DNA
taatacgactcactataggg

SEQ ID NO: 2 - DNA, 25 bp, synthetic construct
  source 1..25: mol_type = other DNA
taatacgactcactatagggataat

SEQ ID NO: 3 - DNA, 19 bp, Escherichia virus T3
  source 1..19: mol_type = genomic DNA
aattaaccctcactaaagg

SEQ ID NO: 4 - DNA, 23 bp, synthetic construct
  source 1..23: mol_type = other DNA
aattaaccctcactaaaggaaga

SEQ ID NO: 5 - DNA, 22 bp, Salmonella virus SP6
  source 1..22: mol_type = genomic DNA
aatttaaggtgacactatagaa

SEQ ID NO: 6 - protein, 877 aa, Escherichia virus T4
  VARIANT 172: note = At this position lysine may be replaced with leucine
  VARIANT 266: note = At this position proline may be replaced with leucine
  VARIANT 772: note = At this position histidine may be replaced with arginine
  VARIANT 748: note = At this position asparagine may be replaced with any amino acid
  VARIANT 756: note = At this position arginine may be replaced with methionine
  VARIANT 758: note = At this position glutamine may be replaced with any amino acid
  VARIANT 775: note = At this position glutamic acid may be replaced with valine
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQWEADMLSKGLLGGEAWSSWHKEDSIHVGVRCIEMLIESTGMVSLHRQNAGWGQDSETIELAPEYAEAIATRAGALAGISPMFQPCWPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEWTVTDENTGEISEKVKLGTKALAGQWLAYGVTRSVTKRSVMTLAYGSKEFGFRQQVLEDTIQPAIDSGKGLMFTQPNQAAGYMAKLIWESVSVTWAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTWWAHEKYGIESFALIHDSFGTIPADAANLFKAVRETMVDTYESCDVLADFYDQFADQLHESQLDKMPALPAKGNLNLRDILESDFAFA

SEQ ID NO: 7 - protein, 128 aa, synthetic construct
FDASNFKDFSSIASASSSWQNQHGSTMIIQVDSFGNVSGQYVNRAEGTGCQNSPYPLTGRVNGTFIDFSVKWNNSTENCNSNTQWTGYAQVNGNNTEIVTRWNLKYEGGSGPAIWQGQDTFQYVPTTE

SEQ ID NO: 8 - DNA, 30 bp, synthetic construct
  source 1..30: mol_type = other DNA
gtgagtggggcaggtggaggtgggagcata

SEQ ID NO: 9 - DNA, 28 bp, synthetic construct
  source 1..28: mol_type = other DNA
wdddywathsractcastawwrrrrvhy

SEQ ID NO: 10 - DNA, 22 bp, synthetic construct
  source 1..22: mol_type = other DNA
wwttavgkgacactatwgrwbd

(19) United States
(12) Patent Application Publication
(10) Pub. No.: US 2025/0034611 A1
(43) Pub. Date: Jan. 30, 2025

(54) Methods and vectors for in vitro transcription of mRNA with reduced levels of dsRNA

(71) Applicant: Applied DNA Sciences, Inc., Stony Brook, NY (US)

(72) Inventors: Yuhua Sun, Stony Brook, NY (US); Minghwa Ben Liang, East Setauket, NY (US); Huan Dong, South Setauket, NY (US); Qiang Zhao, South Setauket, NY (US)

(73) Assignee: Applied DNA Sciences, Inc., Stony Brook, NY (US)

(21) Appl. No.: 18/740,403

(22) Filed: Jun. 11, 2024

Related U.S. Application Data
(60) Provisional application No. 63/472,406, filed on Jun. 12, 2023.

(51) Int. Cl. C12P 19/34; C12N 9/12

(52) U.S. Cl. CPC C12P 19/34; C12N 9/1247; C12Y 207/07006

(57) ABSTRACT

The present application provides improved methods and compositions for in vitro transcription of mRNA that has reduced levels of dsRNA contaminant.

### REFERENCE TO RELATED APPLICATIONS

This application claims the benefit of the filing date of U.S. Provisional application 63/472,406, filed on Jun. 12, 2023, the contents of which is incorporated herein by reference in its entirety.

### INCORPORATION OF SEQUENCE LISTING

The sequence listing in ST.26 XML format entitled 2542-116_ST26.xml, created on Aug. 19, 2024, comprising 13,854 bytes, prepared according to 37 CFR 1.822 to 1.824, submitted concurrently with the filing of this application, is incorporated herein by reference in its entirety.

### FIELD OF THE INVENTION

The present specification relates to improved methods and vectors for the in vitro expression of mRNA that has reduced levels of dsRNA as a contaminant.

### BACKGROUND

In vitro transcription (IVT) using the monomeric bacteriophage DNA Dependent RNA polymerases (RNAPs) (EC 2.7.7.6), is a well established method to generate RNA for a variety of purposes. The RNAP from Escherichia phage T7 (NCBI: txid10760, “Bacteriophage T7” or “Phage T7”) is most commonly used, but homologous RNAPs from Salmonella phage SP6 (NCBI: txid2907955″Bacteriophage SP6″, “Enterobacterial phage SP6”, or “Phage SP6”) and Enterobacteria phage T3 (NCBI: txid10759, “Bacteriophage T3” or “phage T3”) are commercially available. These purposes include mRNA for in vitro translation, mRNA for in vivo translation, RNA probe generation for detection by hybridization and increasingly to generate mRNAs for biopharmaceutical and therapeutic applications. The transcription methods are robust and well established with numerous commercial suppliers providing the necessary components as kits.

Recently the introduction of synthetic in vitro transcribed mRNAs into cells or animal models has revealed undesirable immune effects against the synthetic molecules (Weissman et al. 2000; Karikó et al. 2004; Akira et al. 2006; Sahin et al. 2014; Freund et al. 2019). Immune responses, particularly those in a clinical setting are undesirable and in cases, detrimental.

Double-stranded RNA (dsRNA), present as a contaminant in IVT reactions, has been identified as a major stimulant of the immune response. dsRNA is known to be produced by the bacteriophage polymerases via a variety of mechanisms such as a 3′ extension of the run-off product and through the production of antisense RNAs that can hybridize with the sense strand. Short RNA fragments produced for example during abortive initiation or through degradation can form dsRNA byproducts or serve as templates for RNA synthesis. Bacteriophage RNAPs can also initiate transcription at the ends of DNA templates through a promoter-less mechanism. The causes of the production of dsRNA are complex and depend on a variety of both sequence and non-sequence dependent factors.

A variety of approaches have been sought to reduce or eliminate the immunogenic species in the resulting RNA preparation. Many methods rely on the removal and clean-up of the IVT reaction but these methods are both technically demanding, time consuming, yield reducing, and expensive. Even further, the methods may require optimization for each template.

Other methods to reduce or eliminate immunogenic species rely on changes to the reaction conditions such as modification of the transcription reaction conditions including reducing the Mg2+ concentration and removing the 5′-triphosphate. See Carpousis and Gralla 1980; Schenborn and Mierendorf, 1985; Martin et al. 1988; Konarska and Sharp 1989; Cazenave and Uhlenbeck 1994; Triana-Alonso et al. 1995; Biebricher and Luce 1996; Arnaud-Barbe et al. 1998). Other methods rely on the incorporation of modified nucleosides into the synthetic mRNA. See Karikó et al. 2005; Durbin et al. 2016; Pardi et al. 2017; Richner et al. 2017; Freund et al. 2019). Still other approaches have incorporated high salt conditions and tethering of the template and RNAP on beads (Cavac et al. 2021). Some approaches require individualized optimization of the conditions. In addition to sequence specific effects general mechanisms of dsRNA production and the effects of changing conditions remain incompletely understood. See Rosa et al., (2022).

Study of DNA templates, often linearized plasmids, used for IVT have identified a number of factors that contribute to the production of contaminating species. For example, tt is generally understood to avoid 5′ and 3′ template overhangs. Blunt ends are preferred, however this approach does not eliminate undesirable contaminants. Changes in enzyme: template: NTP conditions can also be used to reduce contaminants, but these approaches can require individualized optimization.

Optimization of the bacteriophage RNAP promoter, generally the T7 bacteriophage promoter, has focused on the sequences within or immediately adjacent to the promoter start site. Generally, phage RNAP promoters comprise a recognition region from −17 to −5 relative to the transcription start site and an initiation region from about −4 to +6. The recognition region includes an AT-rich region from −17 to −13 and a specificity loop between −12 and −5. The initiation region includes an unwinding region from −4 to −1 and the transcription start site at +1 to +6. It is reported that an additional 5 to 10 nucleotides upstream of the promoter (e.g., at −18 to −28) can increase RNA yields. See Tang et al. (2005). These reports do not provide any guidance regarding dsRNA and or other contaminants. Some reports suggest that the overall length of the promoter is about 35 bases. For polymerase amplified IVT templates, the primer includes about 18 bases of target sequence resulting in an amplification primer of about 53 bases or more. See Promega Notes available on the internet at www(dot)Promega(dot)com/resources/pubhub/enotes/important-design-features-of-primers-used-to-generate-dna-template-for-tnt-systems/

The additional steps, decreased yield, and expense, remain challenges to the efficient and safe production of mRNAs, particularly for therapeutic uses. There remains a need for a generalized method to reduce the production of dsRNA and reduce immunogenicity. The present specification identifies the length of the 5′ non-coding region upstream of the bacteriophage promoter as contributing to the production of dsRNA.

### BRIEF SUMMARY

The present application provides for, and includes, a method for producing an RNA composition with reduced levels of dsRNA comprising: synthesizing a deoxyribonucleic acid template (DNA-template) comprising, in order from 5′ to 3′, a 5′-non-coding sequence (NCS): a phage DNA-Dependent RNA Polymerase promoter (phage promoter) sequence; a 5′-untranslated region (UTR), a polypeptide coding sequence (ORF), a 3′ UTR sequence; and 3′ poly(dT) sequence includes more than 100 nucleotides, amplifying the DNA-template using a polymerase chain reaction (PCR) to produce an in vitro transcription template (IVT-template); transcribing the IVT-template in vitro using a phage DNA-Dependent RNA Polymerase transcription system to prepare an RNA composition; wherein the RNA composition has a reduced level of dsRNA compared to an RNA synthesized from a DNA-template without said NCS.

The present application also provides for, and includes, kits for producing an RNA composition with reduced levels of dsRNA comprising: a DNA-template for use in an in vitro transcription reaction; said DNA-template comprising in order from 5′ to 3′: a 5′-non-coding sequence (NCS); a T7 DNA-Dependent RNA Polymerase promoter (T7 promoter) sequence; a 5′-untranslated region (UTR), a polypeptide coding sequence (ORF), and a 3′ UTR sequence, and reagents for performing an in vitro transcription reaction.

The present application also provides for, and includes, a composition, comprising:
DNA-template comprising, in order from 5′ to 3′:
   a. a biotin or avidin at the 5′ end;
   b. a 5′-non-coding sequence (NCS);
   c. a phage DNA-Dependent RNA Polymerase promoter (phage promoter) sequence;
   d. a 5′-untranslated region (UTR),
   e. a polypeptide coding sequence (ORF),
   f. a 3′ UTR sequence; and
   g. 3′ poly(dT) sequence includes more than 100 nucleotides;
PCR components or transcription components and optionally RNA product obtained by in vitro transcription of the DNA-template.

### BRIEF DESCRIPTION OF DRAWINGS

FIG. 1A is a graphical representation of the improved templates of the present application. The figure presents a conventional DNA template having a phage DNA-Dependent RNA Polymerase promoter (phage promoter) sequence, a 5′-untranslated region (UTR), a polypeptide coding sequence (ORF), and a 3′ UTR sequence with an improved template further including a 5′-non-coding sequence (NCS) linked to the phage promoter sequence.

FIG. 1B is a graphical representation of the improved templates of the present application. The figure presents the improved template of FIG. 1A further comprising a 3′ polythymidine sequence.

FIG. 1C is a graphical representation of the improved templates of the present application. The figure presents a double stranded nucleic acid template of FIG. 1B further comprising a 5′ modification. Transcription by a phage polymerase of the templates produces RNA molecules having a 5′-untranslated region (UTR), a polypeptide coding sequence (ORF), and a 3′ UTR sequence (template 1B) optionally with a polyA tail (templates of FIGS. 1B and 1C).

FIG. 2A presents a dot blot detecting dsRNA present in RNA transcribed from the templates of Table 1. In the absence of a 5′ NCS sequence, large amounts of dsRNA are detected (sample 1). The detectable level of dsRNA decreases as the 5′ NCS sequence is increased from 48 to 385 nucleotides (samples 2 to 6). The level of dsRNA is minimized at a 5′ NCS length of 483 and 624 nucleotides (samples 7 and 8) to the level obtained using a circularized plasmid (sample 9).

FIG. 2B presents immunofluorescence of HEK293T cells transfected with the RNA produced from the templates of Table 1.

FIG. 3A presents an exemplary dot blot for the presence of dsRNA according to an embodiment of the present disclosure. As shown, the addition of oligo(dA) to the 5′ end of the DNA templates presented in Table 1 as indicated in Table 2, further reduces the levels of dsRNA detected.

FIG. 3B presents an exemplary dot blot for the presence of dsRNA according to an embodiment of the present disclosure and presents the transfection of the in vitro transcribed mRNA into HEK293T cells and expression of GFP.

FIG. 4A presents a dot blot of dsRNA present in RNA transcribed from the templates of Table 3 that include both a 5′ NCS and a 3′ poly(dT) sequences. Error! Reference source not found.A presents the results of an exemplary dot blot showing that in the presence of a 5′ NCS sequence that is about 100 nucleotides or less, significant amounts of dsRNA are detected (Error! Reference source not found.A, samples 1 and 2). The levels of dsRNA detected decreases as the NCS is increased beyond 289 nucleotides to 893 (Error! Reference source not found.A. samples 3 to 6) to the levels below that obtained using a linearized plasmid (sample 6).

FIG. 4B is a graph showing the relative levels of luciferase expressed from the open reading frame of mRNA transfected into HEK293T cells. The figure presents immunofluorescence of HEK293T cells transfected with the RNA produced from the templates of Table 3.

FIG. 5A is a graphical presentation of RNA yields from an exemplary in vitro transcription reaction for templates F1, F8, F11, F12, F13 and F14. RNA yields are equal to or slightly greater than the linearized plasmid.

FIG. 5B presents a dot blot of dsRNA present in RNA transcribed from the templates of Table 4.

FIG. 6 presents a diagram of T7 RNA Polymerase binding sites (SEQ ID NO:9).

FIG. 7 presents a diagram of SP6 RNA Polymerase binding sites showing the preferred nucleotides (SEQ ID NO:10).

### DETAILED DESCRIPTION

The present specification provides for, and includes, methods for producing an RNA composition with reduced levels of dsRNA comprising synthesizing a deoxyribonucleic acid template (DNA-template) comprising, in order from 5′ to 3′, a 5′-non-coding sequence (NCS), a phage DNA-Dependent RNA Polymerase promoter (phage promoter) sequence, a 5′-untranslated region (UTR), a polypeptide coding sequence (ORF), a 3′ UTR sequence, and 3′ poly(dT) sequence, amplifying the DNA-template using a polymerase chain reaction to produce an in vitro transcription template (IVT-template), transcribing the IVT template in vitro using a phage DNA-dependent RNA polymerase transcription system to prepare and RNA composition that has reduced levels of double-stranded RNA (dsRNA) compared to an RNA composition synthesized from a DNA-template that lacks a 5′ NCS.

### The DNA-Template

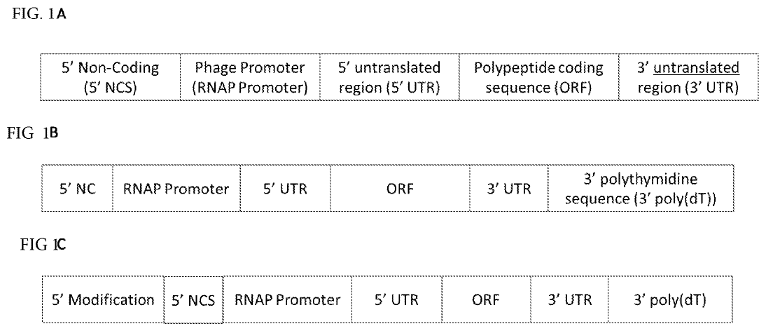

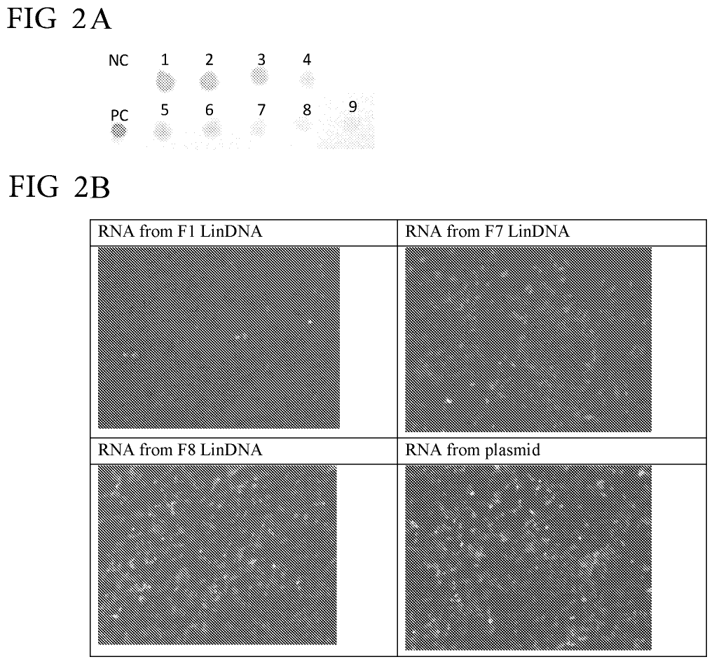

The DNA-templates of the present disclosure are illustrated graphically in FIG. 1. As shown in FIG. 1A, the template comprises a number of conventional features found in transcription templates for the expression of a protein sequence of interest. At the core, is a polypeptide encoding sequence presented as an open reading frame (ORF). The ORF is followed by a 3′ untranslated region (3′ UTR) that is known to enhance RNA stability and to increase protein expression. Linked at the 5′ end of the ORF is a 5′ untranslated region (5′ UTR) providing at least the function of providing a ribosomal binding site. Transcription is driven from a phage promoter, typically about 35 nucleotides in length. As shown in FIG. 1B, the 3′ UTR can further include a 3′ Poly(dT) sequence.

### 5′ NCS

The methods of the present specification provide for, and include, a 5′ non-coding sequence (NCS) that is located 5′ to a phage promoter sequence and acts to reduce the amount of undesirable dsRNA produced during in vitro transcription and increase protein expression in cells. As shown in Example 6, in the absence of a 5′ NCS sequences, high levels of dsRNA are detected. As shown, even a short 5′ NCS sequence of 48 to 103 nucleotides decreases the amount of dsRNA in the RNA composition relative to a DNA template lacking a 5′ NCS. As the length of the 5′ NCS is increased between 185 to 385 nucleotides, an additional reduction of dsRNA is observed relative to the shorter 5′ NCS sequences. As the 5′ NCS is further increased, the dsRNA is further reduced.

In aspects, the 5′ NCS of the DNA-templates of the present specification is at least 150 nucleotides in length. In aspects, the length of the 5′ NCS sequence is at least 350 nucleotides in length. In other aspects, the length of the 5′ NCS sequence is at least 500 nucleotides in length. The addition of additional NCS sequences beyond 500 nucleotides result in similar levels of dsRNA. While longer NCS sequences are possible (e.g., >500 nucleotides) and included in the methods of the present specification, increasing the overall length of the DNA-template incurs an increased cost and are often avoided. In aspects, the NCS sequences of the DNA-templates of the present application are between 500 and 1000 nucleotides.

Not to be limited by theory, it is believed that the presence of the additional NCS sequences provides for a binding region for the RNA polymerase where the RNAP remains inactive for dsRNA production. The binding of the RNAP to the NCS region is thought to decrease the level of RNAP available to catalyze side reactions such as 3′ extension of the product, production of anti-sense RNAs. and other promoter independent reactions. Thus it is thought that the sequence of the NCS plays a minimal role in the reduction of dsRNA.

### 5′ Modification

In aspects of the present specification, the end of the 5′ NCS sequence can be modified to incorporate ligand, typically a biotin moiety. The presence of a ligand such as biotin allows for the immobilization of the DNA-template to a substrate or for interacting with a phage polymerase fusion protein comprising a ligand (e.g., biotin) binding domain as detailed below. In aspects, the phage RNAP is fused to an avidin, including but not limited to streptavidin, rhizavidin, and neutravidin, such as Extravidin®, NeutrAvidin®, or NeutraLite. The combination of the 5′ biotin moiety and the phage RNAP fusion protein increases the yield of RNA during the transcription process compared to traditional phage RNA polymerases.

In other aspects, methods including the 5′ biotin modification and phage RNAP fusion proteins provides for a reduction in the amount of DNA template necessary for the transcription reaction.

### Promoters and RNAP

Phage DNA-dependent RNA polymerase promoters and phage DNA-Dependent RNA Polymerase transcription systems are known in the art. Promoter/transcription systems based on T7 are most commonly used, but promoter/transcription derived from T3 phage, SP6 phage, and others are suitable for use in the present methods. An advantage of the phage transcription system is the single chain DNA-dependent RNA polymerase having a high specificity for promoters having a length of about 23 nucleotides though additional sequences at the promoter can influence initiation. See Ikeda and Richardson 1986. In some applications, an additional five to ten bases is recommended to aid transcription initiation and promoter strength. See Promoga notes; Tang et al. 2005; and Baklanov et al. 1996.

In aspects, the phage DNA-dependent RNA polymerase promoter and phage DNA-Dependent RNA Polymerase transcription system is derived from Escherichia phage T7, Salmonella phage SP6, or Enterobacteria phage T3. While the T7, T3, and SP6 phages are the most well known, other phage polymerase transcription systems are suitable for use in the methods of the present disclosure. In aspects, other promoter and phage transcription systems can be obtained from the Autographiviridae bacteriophage family including teseptimavirus (NCBI: txid110456), Zindervirus (NCBI: txid542837), or Teetrevirus (NCBI: txid2732693), species. As used herein, a phage transcription system refers to the combination of the phage promoter and the phage DNA-dependent RNA polymerase (“RNAP”).

### T7 RNAP System

In aspects, the phage transcription system is a T7 phage transcription system comprising a T7 promoter and T7 RNAP. In aspects, the T7 promoter comprises the sequences represented by the logo as shown in the Error! Reference source not found. (See online at parts(dot)igem(dot)org/Promoters/Catalog/T7). See Pribnow D. Nucleotide sequence of an RNA polymerase binding site at an early T7 promoter. Proc Natl Acad Sci USA. 1975 March; 72 (3): 784-8. doi: 10.1073/pnas.72.3.784. PMID: 1093168; PMCID: PMC432404.

In aspects, an exemplary core T7 promoter comprises the sequence TAATACGACTCACTATAGGG (SEQ ID NO: 1) wherein the transcription start site is underlined. Provided for, and included, in the methods of the present specification are variations of the exemplary promoter that are known to increase the transcription efficiency. In aspects, the T7 promoter is an optimized promoter comprising the sequence TAATACGACTCACTATAGGGATAAT (SEQ ID NO:2). Sequences from positions +4 to +8 downstream of the transcription start site can affect promoter activity over a 5-fold range. See Conrad et al. 2020. Mutations between −17 and +3 can affect transcription efficiency and are provided for, and included, in the present specification. Imburgio et al., Studies of Promoter Recognition and Start Site Selection by T7 RNA Polymerase Using a Comprehensive Collection of Promoter Variants. https://doi.org/10.1021/B1000365W (2000); Patwardhan, R. P. et al. High-resolution analysis of DNA regulatory elements by synthetic saturation mutagenesis. Nat. Biotechnol. 27, 1173-1175 (2009); Komura, R., Aoki, W., Motone, K., Satomura, A. & Ueda, M. High-throughput evaluation of T7 promoter variants using biased randomization and DNA barcoding. PLOS ONE 13, e0196905 (2018). T7 promoters of the present specification also include additions upstream of the core T7 promoter that increase promoter strength. See Tang, G.-Q., Bandwar, R. P. & Patel, S. S. Extended upstream A-T sequence increases T7 promoter strength. J. Biol. Chem. 280, 40707-40713 (2005).

The present specification provides for, and includes, transcribing the 5′ NCS containing IVT-templates in vitro using a phage DNA-Dependent RNA Polymerase transcription system. T7 phage RNAP (NCBI Reference Sequence: NP_041960.1) suitable for use in the methods of the present specification are known in the art and widely available. See WO 00/36112.

In aspects, the phage DNA-Dependent RNA Polymerase is a T7 DNA-Dependent RNA polymerase (“T7 RNAP”) that is a fusion protein, including for example the T7 RNAP fusion proteins as described in International Patent Publication No. WO 2022/084748, herein incorporated by reference in its entirety. In an aspect, the T7 RNAP is fusion protein comprising an RNA polymerase or a functional fragment thereof and a binding domain. In aspects, the T7 RNAP fusion protein comprises T7 RNAP and a DNA binding domain. In other aspects, the T7 RNAP fusion protein comprises T7 RNAP and a ligand binding domain. In aspects the ligand binding domain binds to biotin. In some instances, the ligand binding domain comprises an avidin. Non-limiting examples of an avidin include streptavidin, rhizavidin, and neutravidin, such as Extravidin®, NeutrAvidin®, or NeutraLite. In some embodiments, the ligand binding domain is a Streptavidin. In some embodiments, the ligand binding domain is a monomeric streptavidin. In some embodiments, the ligand binding domain is rhizavidin. In some embodiments, the DNA binding domain comprises a sequence comprising SEQ ID NO:7.

### T3 RNAP System

In aspects, the phage transcription system is a T3 phage transcription system comprising a T3 promoter and T3 RNAP. In aspects, an exemplary T3 promoter comprises the sequence AATTAACCCTCACTAAAGG (SEQ ID NO: 3). Like T7, transcription efficiency of T3 promoters is affected by sequences both immediately upstream and downstream of the core sequence. In an aspect, the T3 promoter comprises the sequence AATTAACCCTCACTAAAGGAAGA (SEQ ID NO:4). Like the T7 RNAP of the phage transcription system, T3 RNAPs suitable for use in the present methods are known in the art. T7 and T3 RNAPs share significant homology and homologous mutations are known to share properties. For example, T7 RNAP having a serine to proline mutation at position 633 and the corresponding serine to proline mutation at position 634 in T3 RNAP increases RNAP stability. See WO WO 00/36112. Similarly, the present methods provide for, and include, fusion proteins of T3 RNAP homologous to the fusion proteins of T7 RNAP as described in WO 2022/084748, and discussed above. More specifically, in aspects of the present methods, the T3 RNAP is fusion protein comprising an RNA polymerase or a functional fragment thereof and a binding domain. In aspects, the T3 RNAP fusion protein comprises T3 RNAP and a DNA binding domain. In other aspects, the T3 RNAP fusion protein comprises T3 RNAP and a ligand binding domain. In aspects the ligand binding domain binds to biotin. In some instances, the ligand binding domain comprises an avidin. Non-limiting examples of an avidin include streptavidin, rhizavidin, and neutravidin, such as Extravidin®, NeutrAvidin®, or NeutraLite. In some embodiments, the ligand binding domain is a Streptavidin. In some embodiments, the ligand binding domain is a monomeric streptavidin. In some embodiments, the ligand binding domain is rhizavidin. In some aspect, the DNA binding domain comprises a sequence comprising SEQ ID NO: 7.

### SP6 RNAP System

In aspects, the phage transcription system is a SP6 phage transcription system comprising a SP6 promoter and SP6 RNAP. In aspects, an exemplary SP6 promoter comprises the sequence AATTTAAGGTGACACTATAGAA (SEQ ID NO: 5). See Shin, I., Kim, J., Cantor, C. R. & Kang, C. Effects of saturation mutagenesis of the phage SP6 promoter on transcription activity, presented by activity logos. Proc. Natl Acad. Sci. USA 97, 3890-3895 (2000). In aspects, the SP6 promoter comprises the sequences represented by the logo as shown in Error! Reference source not found.

### Untranslated Region UTR

The methods of the present specification provide for, and include, untranslated regions located 5′ and 3′ to the protein coding sequences. mRNAs in vivo generally include untranslated regions (UTRs) on either or both sides of the coding sequence. If positioned on the 5′ side, it is called a 5′ UTR (or leader sequence), or if positioned on the 3′ side, it is called a 3′ UTR (or trailer sequence).

UTRs can have a variety of biological functions, including but not limited to translation regulation, ribosome entry, mRNA localization, mRNA export, stability regulation, as well as providing regulatory targets for trans acting factors such as micro-RNAs, and RNA-binding proteins. A 5′ UTR can form secondary structures that regulate translation and generally comprises a sequence that is recognized by the ribosome that allows the ribosome to bind and initiate translation of the mRNA. Some 5′ UTRs have been found to interact with proteins and some 5′ UTR sequences have been linked to mRNA localization and export signals and cellular mechanisms. Similarly, the sequences and structures of 3′-untranslated regions (3′ UTRs) of messenger RNAs can serve to regulate the gene expression, for example by affecting mRNA stability, localization, and expression. 3′ UTR regulatory elements are recognized by a wide variety of trans-acting factors that include microRNAs (miRNAs), their associated machinery, and RNA-binding proteins (RBPs). In turn, these factors instigate common mechanistic strategies to execute the regulatory programs that are encoded by 3′ UTRs.

The templates of the present specification include a 5′ untranslated region located 3′ to the phage promoter sequences. That is, the 5′ UTR becomes part of the RNA transcript and is located at the 5′ end of the RNA. As provided herein, the selection of the 5′ UTR of the present templates is determined by the purposes of the mRNA. In aspects, the 5′ UTR comprises the native or homologous 5′ UTR of the polypeptide coding sequence. By retaining the native 5′ UTR, the mRNA present in the RNA composition produced can retain the native regulation of the polypeptide. In other aspects, a heterologous, or non-native 5′ UTR can be used. In general, a 5′ UTR sequence would retain the ribosome binding sequences to ensure initiation of translation. In other aspects, regulatory signals present in a heterologous 5′ UTR can be incorporated. The selection of the appropriate 5′ UTR for use in a DNA template is within the skill of a person of ordinary skill in the art. See Cao et al., “High-throughput 5′ UTR engineering for enhanced protein production in non-viral gene therapies,” Nat Commun 12, 4138 (2021). Doi(dot)org/10.1038/s41467-021-24436-7

### Polypeptide Coding Sequence

The methods of the present specification provide for, and include, a polypeptide encoding sequence that provides an open reading frame (ORF) for a polypeptide of interest. In some aspects, the 5′ UTR is homologous to the ORF. Suitable polypeptide coding sequences are known in the art and can be found in a variety of public databases including but not limited to the National Library of Medicine NCBI databases.

### 3′ UTR Sequence

As provided herein, the DNA templates of the present specification include an untranslated region located 3′ from the polypeptide coding sequence. In aspects, the 3′ UTR sequence can be homologous to the polypeptide coding sequence or heterologous.

3′ poly(dT) Sequence

As provided herein, the DNA templates of the present specification include a 3′ poly(dT) sequence. In aspects, the 3′ poly(dT) sequence comprises at least 50 bases. In an aspect the 3′ poly(dT) sequence comprises at least 75 bases. In an aspect the 3′ poly(dT) sequence comprises at least 100 bases. In an aspect the 3′ poly(dT) sequence comprises at least 120 bases. In certain aspects, the DNA template lacks a 3′ poly(dT) sequence and polyA sequences, necessary for optimal protein expression is added to the in vitro transcribed transcript.

### PCR Amplification

The present specification provides for, and includes, a step of preparing an IVT-template using the polymerase chain reaction (PCR). The methods and materials for PCR are known in the art and comprise multiple cycles that may comprise a denaturing step, an annealing step, and an extension step. In some embodiments, each PCR cycle may comprise a denaturing step and an extension step. Generally, the methods provide for between 10 and 50 cycles of PCR to amplify the IVT-template in sufficient quantity to serve as a transcription template.

### EXAMPLES

### Example 1: In Vitro Transcription of DNA In Vitro Transcription (IVT) Template

DNA templates are prepared via PCR as provided herein. Alternatively, the DNA templates may be conjugated with biotin at the 5′ of the non-template strand. Real-time RNA polymerization assays are initiated by adding a T7 RNA polymerase or a modified fusion RNA polymerase comprised of DNA binding domain (e.g. Rhizavidin) and a T7 RNA polymerase into 20 microliters (μl) of the reaction mixture containing 100 μM DFHBI-1 T, 400 nM template, 4 mM NTPs, 0.1 U inorganic pyrophosphatase and 1× transcription buffer. The reaction mixture is incubated for up to 1 hour at 37° C. and the enzyme activity is measured.

### Example 2: Detection of dsRNA Production

RNA produced using the method of Example 3 is analyzed by dot blot according to the method of Baiersdörfer et al. “A Facile Method for the Removal of dsRNA Contaminant from In Vitro-Transcribed mRNA,” Mol Ther Nucleic Acids 15:26-35 (2019). Briefly, 2 micrograms (μg) of total RNA from an in vitro transcription reaction are spotted on a positively charged nylon membrane (Whatman Nytran SuPerCharge, Sigma-Aldrich). Sample leakage is prevented using a silicone mask (Bio-Dot Gasket 96 wells. Bio-Rad, Munich, Germany). After loading, the sealing gasket is removed and the air-dried membrane transferred to a 50-mL tube and blocked in 5% (w/v) non-fat dried milk in Tris-buffered saline (TBS)-T buffer (20 mM Tris [pH 7.4], 150 mM NaCl, and 0.1% (v/v) Tween-20).

dsRNA is detected by incubating the membrane with J2 anti-dsRNA murine antibody (Scicons, Budapest, Hungary) diluted 1:5,000. RNA:DNA hybrids are detected with S9.6 murine monoclonal antibody (mAb) (Kerafast, Boston, MA. USA) diluted 1:20,000. Hybridization and washing is performed using standard methods and antibody binding was detected by incubating the membrane with horseradish peroxidase (HRP)-conjugated donkey antimouse immunoglobulin G (IgG) (Jackson ImmunoResearch Laboratories, Cambridgeshire, UK) diluted 1:10,000. After washing the membranes using standard methods, chemiluminescence detection is performed using Amersham ECL Prime Western Blotting Detection Reagent (GE Healthcare Life Sciences) and the ChemiDoc MP Imaging System (Bio-Rad). The signal intensities of the dots are quantified by densitometry using the Volume Tools of the Image Lab software (Bio-Rad). Equal sample loading is verified by stripping the membranes with 1% (w/v) SDS containing 40 mM dithiothreitol at 60° C. for 30 min and probed with a 3′-biotinylated oligodeoxynucleotide (5′-GTG AGT GGG GCA GGT GGA GGT GGG AGC ATA-3′) (SEQ ID NO:8) complementary to the 3′ UTR of the mRNA. Reprobing with oligodeoxynucleotide (ODN) is performed in PerfectHyb Plus Hybridization Buffer (Sigma-Aldrich), according to the manufacturer, and HRP-conjugated streptavidin (Thermo Fisher Scientific) is used for visualization.

### Example 3: Upstream DNA Sequences Reduce dsRNA and Increase Protein Expression

DNA in vitro transcription (IVT) templates are prepared as provided in Example 1 and presented in Table 1.

TABLE 1
IVT templates

| | IVT | | Non-coding | |
|---|---|---|---|---|
| | template | | sequence length | |
| Sample | length | ORF | (bp) | dsRNA |
| F1 | 867 | GFP | n/a | ++++ |
| F2 | 915 | GFP | 48 | +++ |
| F3 | 970 | GFP | 103 | +++ |
| F4 | 1052 | GFP | 185 | ++ |
| F5 | 1156 | GFP | 289 | ++ |
| F6 | 1252 | GFP | 385 | + |
| F7 | 1350 | GFP | 483 | + |
| F8 | 1491 | GFP | 624 | + |
| Linearized Plasmid | | GFP | 2866 | + |
| Negative control | n/a | n/a | n/a | − |
| (H2O)C | | | | |
| dsRNA control | n/a | n/a | n/a | |

Templates are amplified as provided herein, and transcribed in vitro as provided in Example 1. dsRNA is detected as provided in Example 2 using 2.4 μg RNA for each sample. The results are presented in Error! Reference source not found.A.

The GFP expression profile of in vitro RNA from sample F1, F7, F8 and plasmid are presented in Error! Reference source not found.B. As shown in Error! Reference source not found.B, RNA produced from sample F8 having a non-coding sequence length (bp) of 624 base pairs is indistinguishable from RNA produced from a linearized plasmid. Very low levels of GFP expression is detected from Sample F1 lacking a non-coding sequence 5′ to the T7 RNAP promoter. Good expression is obtained from sample F7 having a non-coding sequence length of 483 bp, but less than the level of expression obtained from Sample F8 and linearize plasmid.

### Example 4: PolyT Reduces dsRNA Production and Increases Protein Expression

DNA in vitro transcription (IVT) templates are prepared as provided herein and further comprising a 3′-poly(dT) sequence and presented in Table 2.

TABLE 2
IVT templates having 3′-polydT sequence

| | | IVT | | | Non-coding | |
|---|---|---|---|---|---|---|
| | Sample | template | | | sequence | |
| Sample | name | length | polydT | ORF | length (bp) | dsRNA |
| 1 | F1T | 981 | 114 | GFP | n/a | ++ |
| 2 | F4T | 1166 | 114 | GFP | 185 | ++ |
| 3 | F6T | 1366 | 114 | GFP | 385 | − |
| 4 | F8T | 1605 | 114 | GFP | 624 | +/− |
| 5 | F11T | 1874 | 114 | GFP | 893 | − |
| 6 | Linearized | | | GFP | 2866 | ++ |
| | Plasmid | | | | | |
| | Negative | n/a | | n/a | n/a | − |
| | control | | | | | |
| | (H2O)C | | | | | |
| | dsRNA | n/a | | n/a | n/a | ++ |
| | control | | | | | |

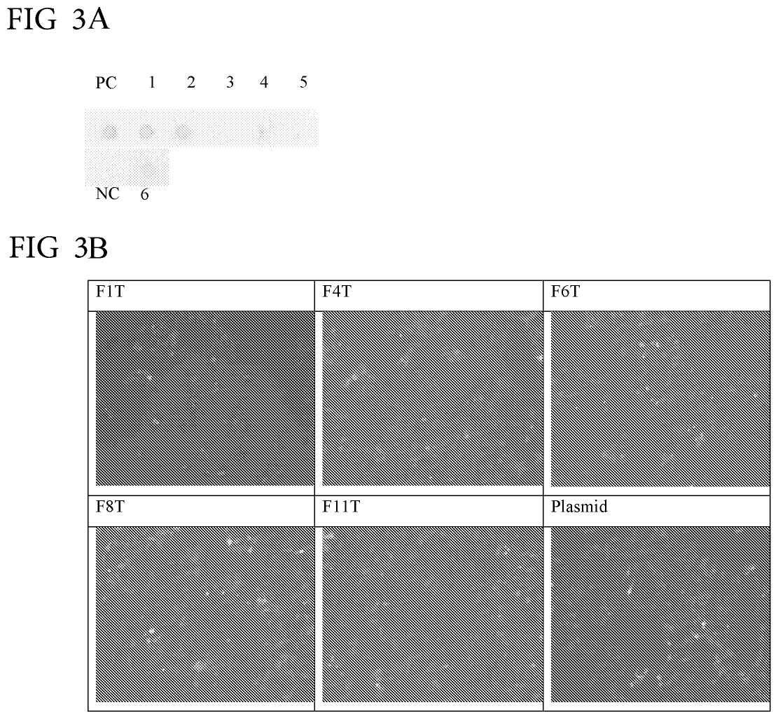

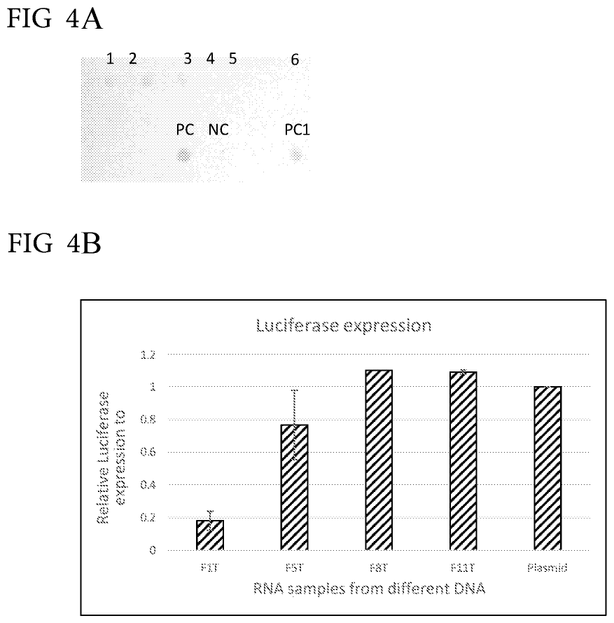

Templates are amplified via PCR as provided herein, and transcribed in vitro as provided in Example 1. Upon transcribing the templates, the resulting RNA sequences include a polyA tail (e.g., the reverse complement of the 3′-poly(dT) sequences. dsRNA is detected as provided in Example 2 using 2.0 μg RNA for each sample. The results are presented in Error! Reference source not found.A. As shown, including 3′-poly(dT) sequences in the template further decreases the levels of dsRNA detected in the RNA composition over the levels present in transcription reactions with the 5′-NCS sequences alone. As shown in samples 3, 4, and 5, of FIG. 3B, the presence of dsRNA is largely eliminated. As shown in Error! Reference source not found.A, the presence of both an NCS sequence and a 3′ poly(dA) sequence in the template greatly reduces the level of dsRNA.

The GFP expression profile of in vitro RNA from sample FIT, F4T, F6T, F8T, F11T, and plasmid, are presented in Error! Reference source not found.B. As shown in Error! Reference source not found.B, RNA produced from samples F8T and F11T presented similar expression profiles to the produced by RNA from a linearized plasmid. RNA produced from IVT template FIT exhibits lower levels of expression of GFP. Thus, the inclusion of the 5′ NCS sequences and 3′ poly(dA) sequences act to reduce the levels of dsRNA while not compromising the translation efficiency of the resulting mRNA.

### Example 5

DNA in vitro transcription (IVT) templates are prepared as provided herein and presented in Table 3.

TABLE 3
IVT templates having 3′ polyT sequence for Luciferase expression

| | | | | 5′ Non- | |
|---|---|---|---|---|---|
| | | IVT | | coding | |
| | | template | | sequence | |
| Sample | Sample Name | length | ORF | length (bp) | dsRNA |
| 1 | F1T-Luc | 1830 | Luciferase | n/a | ++ |
| 2 | F3T-Luc | 1933 | Luciferase | 103 | ++ |
| 3 | F5T-Luc | 2119 | Luciferase | 289 | − |
| 4 | F8T-Luc | 2454 | Luciferase | 624 | +/− |
| 5 | F11T-Luc | 2723 | Luciferase | 893 | − |
| 6 | Linearized | | Luciferase | 2866 | ++ |
| | Luciferase | | | | |
| | Plasmid | | | | |
| NC | Negative | n/a | n/a | n/a | − |
| | control | | | | |
| | (H2O)C | | | | |
| PC | dsRNA control | n/a | n/a | n/a | ++ |

The linear DNA templates differ from the templates of Example 3 by substitution of the green fluorescent protein (GFP) with an open reading from for luciferase. As shown in Error! Reference source not found.A, similar reductions of dsRNA are observed demonstrating that the sequence of the ORF does not significantly effect the reduction in dsRNA. Templates are PCR amplified as provided herein, and transcribed in vitro as provided in Example 2. dsRNA is detected as provided in Example 4 using 1.0 μg RNA for each sample. The results are presented in Error! Reference source not found.A. dsRNA is detectable in IVT sample FIT-Luc and F3T-Luc.

The luciferase expression profile of in vitro RNA from sample FIT-Luc, F5T-uc, F8T-Luc, F11T-Luc, and plasmid, are presented in Error! Reference source not found.B. As shown in Error! Reference source not found.B, RNA produced from samples FIT-Luc and F5T-Luc present reduced luciferase expression. Luciferase expressed from sample F8T-Luc is similar to further extended IVT temple F11T-Luc and linearize plasmid.

### Example 5

DNA in vitro transcription (IVT) templates are prepared as provided herein and presented in Table 4. The sequence of the IVT templates in Example 4 were modified to be compatible with the CleanCap AG co-transcriptional capping reagents, which after IVT, results in a 5′ capped mRNA with a Cap1 structure.

TABLE 4
IVT templates having 3′ polyT sequence for Luciferase expression
with CleanCap AG co-transcriptional 5′ capping

| | | | 5′ Non- | |
|---|---|---|---|---|
| | | | coding | |
| | | | sequence | |
| Sample | Sample Name | ORF | length (bp) | dsRNA |
| 1 | F1 | Luciferase | 44 | ++ |
| 2 | F8 | Luciferase | 565 | ++ |
| 3 | F11 | Luciferase | 828 | ++ |
| 4 | F12 | Luciferase | 1300 | + |
| 5 | F13 | Luciferase | 2159 | − |
| 6 | F14 | Luciferase | 2767 | − |
| 7 | Linearized Luc | n/a | n/a | − |
| | Plasmid | | | |
| PC | dsRNA control | n/a | n/a | +++ |

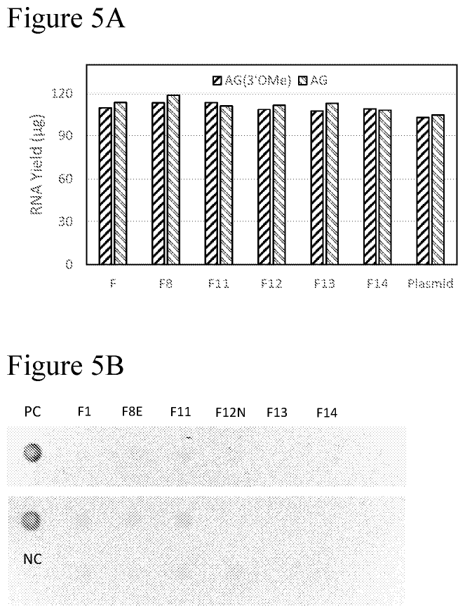

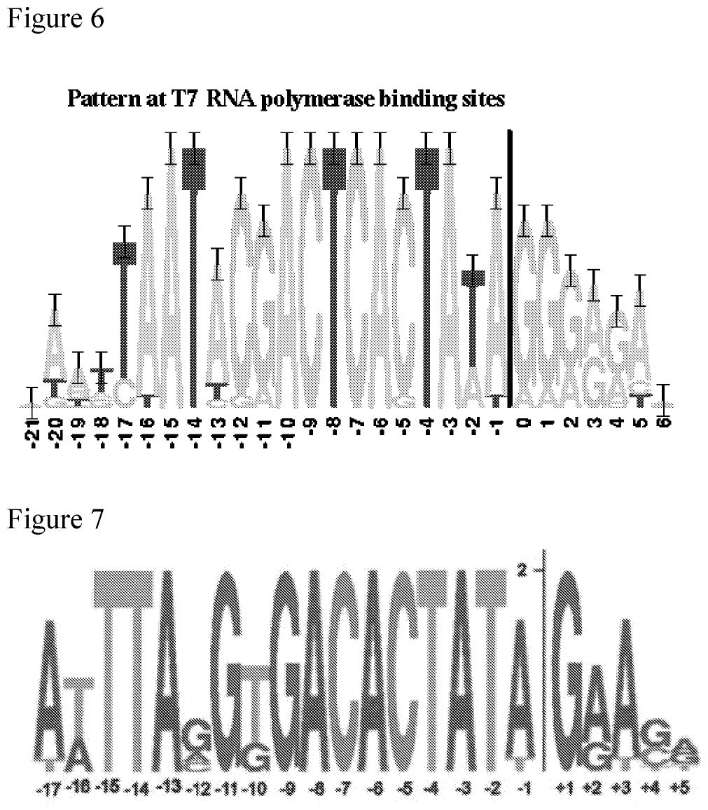

IVT-based production of 5′ capped mRNA was performed in accordance with Example 1, using CleanCao AG Reagent (TriLink Biotechnologies, USA) according to manufacturer instructions. dsRNA was detected according to Example 2, using 1.0 μg RNA for each sample. As shown in FIG. 5B, dsRNA contamination in samples F12, F13 and F14 was less than the dsRNA contamination found in samples F1, F8 and F11, which contained shorter non-coding sequences. This shows that liked non-capped mRNA (Examples 3-5), extended non-coding sequences also reduce the amount of dsRNA contamination in 5′ capped mRNA.

The amount of RNA produced by the IVT reactions was not materially altered by the non-coding sequences of samples F1, F8, F11, F12, F13 and F14. As shown in FIG. 5A, F1, F8, F11, F12, F13 and F14 RNA yields were equal to or slightly greater than the linearized plasmid.

1. A method for producing an RNA composition with reduced levels of dsRNA comprising:
synthesizing a deoxyribonucleic acid template (DNA-template) comprising, in order from 5′ to 3′:
a. a 5′-non-coding sequence (NCS);
b. a phage DNA-Dependent RNA Polymerase promoter (phage promoter) sequence;
c. a 5′-untranslated region (UTR),
d. a polypeptide coding sequence (ORF),
e. a 3′ UTR sequence; and
f. 3′ poly(dT) sequence includes more than 100 nucleotides.
amplifying said DNA-template using a polymerase chain reaction (PCR) to produce an in vitro transcription template (IVT-template);
transcribing said IVT-template in vitro using a phage DNA-Dependent RNA Polymerase transcription system to prepare an RNA composition;
wherein said RNA composition has a reduced level of dsRNA compared to an RNA synthesized from a DNA-template without said NCS.

2. The method of claim 1, wherein said non-coding sequence (NCS) is at least 500 nucleotides in length.

3. The method of claim 1, wherein said DNA-template further comprises a biotin moiety attached to the 5′ NCS.

4. The method of claim 3, wherein said phage DNA-Dependent RNA Polymerase bacteriophage transcription system comprises a modified T7 RNA polymerase fused to a DNA binding domain selected from streptavidin, rhizavidin, or neutravidin, or modified forms thereof.

5. The method of claim 1, wherein said reduced level of dsRNA contamination comprises less than 1% of the total amount of mRNA synthesized.

6. The method of claim 1, further comprising capping the RNA in said RNA composition.

7. The method of claim 2, wherein said NCS comprises:
a. between 500 and 1000 nucleotides;
b. a G/C content of between 45% and 55%.

8. The method of claim 2, wherein said NCS does not comprise a sequence selected from
a. an origin of replication;
b. a phage origin of replication;
c. a polylinker;
d. an open reading frame encoding an antibiotic resistance polypeptide;
e. a transposon sequence; or
f. an open reading frame encoding a LacZ gene or fragment thereof.

9. The method of claim 1, wherein said DNA-template is synthesized in vitro.

10. The method of claim 9, wherein said DNA-template is synthesized as a linear double stranded, blunt ended molecule.

11. A kit for producing an RNA composition with reduced levels of dsRNA comprising:
in order from 5′ to 3′:
a DNA-template for use in an in vitro transcription reaction; said DNA-template comprising
a. a 5′-non-coding sequence (NCS);
b. a T7 DNA-Dependent RNA Polymerase promoter (T7 promoter) sequence;
c. a 5′-untranslated region (UTR),
d. a polypeptide coding sequence (ORF),
e. and a 3′ UTR sequence; and
reagents for performing an in vitro transcription reaction.

12. The kit of claim 11, wherein said bacteriophage DNA-Dependent RNA Polymerase is T7 bacteriophage DNA-Dependent RNA Polymerase, an SP6 bacteriophage DNA-Dependent RNA Polymerase, a T3 bacteriophage DNA-Dependent RNA Polymerase, or functional fragments of any.

13. The kit of claim 12, wherein said T7 RNA polymerase or a functional fragment thereof, wherein the variant T7 RNA polymerase comprises at least four variations selected from the group consisting of K172L, P266L, H772R, N748X, R756M, Q758X, and E775V, wherein the position is determined by alignment with SEQ ID NO: 6, and wherein X is any amino acid different from the wild type amino acid.

14. The kit of claim 11, wherein said DNA IVT template further comprises a 3′ polythymidine sequence (3′ poly(dT)) located 3′ to said 3′ UTR sequence.

15. The kit of claim 12, wherein said 3′ poly(dT) sequence includes more than 100 nucleotides.

16. The kit of claim 11, further comprising buffers, reagents, and enzymes for amplifying said DNA IVT template using the polymerase chain reaction (PCR).

17. The kit of claim 11, further comprising buffers, reagents, and enzymes for transcribing RNA from a PCR amplified DNA IVT template.

18. The kit of claim 17, wherein said enzyme is T7 DNA Dependent RNA Polymerase, SP6 DNA Dependent RNA Polymerase, or T3 DNA Dependent RNA Polymerase.

19. The kit of claim 18, wherein said enzyme is T7 DNA Dependent RNA Polymerase.

20. A composition, comprising:
DNA-template comprising, in order from 5′ to 3′:
a. a biotin or avidin at the 5′ end;
b. a 5′-non-coding sequence (NCS);
c. a phage DNA-Dependent RNA Polymerase promoter (phage promoter) sequence;
d. a 5′-untranslated region (UTR),
e. a polypeptide coding sequence (ORF),
f. a 3′ UTR sequence; and
g. 3′ poly(dT) sequence includes more than 100 nucleotides;
PCR components or transcription components optionally RNA product.
